(19) United States
(12) Patent Application Publication
(10) Pub. No.: US 2025/0186075 A1
(43) Pub. Date: Jun. 12, 2025

(54) TISSUE MANIPULATION AND CUTTING DEVICE AND RELATED METHODS

(71) Applicant: Wisconsin Alumni Research Foundation, Madison, WI (US)

(72) Inventors: Molly Paras, Pewaukee, WI (US); Zachary Spears, Eden Prairie, MN (US); Peter Wawrzyn, Delafield, WI (US); Logan De Lacy, Bettendorf, IA (US); Srihari Gopalan, Verona, WI (US); Kyle Muckenhirn, Madison, WI (US); Katriel Tan Williams, Great Neck, NY (US); Madeline Johnson, Eden Prairie, MN (US); Maggie Anderson, Edina, MN (US); Alexis Block, Chanhassen, MN (US); Jason Hahn, Woodland Hills, CA (US); Noah Hamrin, Glendale, WI (US); Emma Flemmer, Chaska, MN (US); John Puccinelli, Cottage Grove, WI (US); Samuel Conway, Onalaska, WI (US); Lauren Fitzsimmons, Mequon, WI (US)

(21) Appl. No.: 18/972,024

(22) Filed: Dec. 6, 2024

Related U.S. Application Data
(60) Provisional application No. 63/607,999, filed on Dec. 8, 2023.

(51) Int. Cl. A61B 17/295; A61B 17/29; A61B 17/32; A61B 17/3201

(52) U.S. Cl. CPC A61B 17/295; A61B 17/3201; A61B 2017/2929; A61B 2017/320052

(57) ABSTRACT

Combination tissue manipulation and cutting devices having first and second elongate members rotatably coupled to each other, a blade guide housing disposed on one of the elongate members, and a blade drive body slidably disposed in the blade guide housing and having a blade attachment component. Some blade drive body embodiments include a detent disposed on the body. Certain device embodiments including the first elongate member having a guide slot defined therein such that the blade drive body is slidably disposed adjacent to or within the guide slot. Also provided are methods of manipulating and cutting tissue that include engaging target tissue with any surgical device embodiment herein and urging the blade drive body and blade distally through the blade guide housing such that the blade cuts the target tissue.

### CROSS-REFERENCE TO RELATED APPLICATION(S)

This application claims the benefit under 35 U.S.C. § 119 (e) to U.S. Provisional Application 63/607,999, filed Dec. 8, 2023 and entitled “Tissue Manipulation and Cutting Device and Related Methods,” which is hereby incorporated herein by reference in its entirety.

### FIELD

The various embodiments herein relate to handheld surgical devices, and more specifically to devices for grasping, manipulating, and/or cutting tissue during a surgical procedure.

### BACKGROUND

Various conditions require a surgical approach that includes navigation between tissue planes and dissection of one tissue without cutting or damaging surrounding tissues. For example, cubital tunnel syndrome results from damage to the ulnar nerve in the cubital tunnel (the area of the elbow), often resulting from compression of the nerve by nearby structures, including tight fascia bands.

The process of repairing the ulnar nerve (called “ulnar nerve decompression surgery”) requires navigation between the tissue planes and dissection of the fascia band that is overlaying the nerve to release the nerve and restore blood flow. One of the most common techniques for the decompression surgery is an open procedure in which a large incision is required to expose the entire nerve followed by insertion of two different instruments (a forceps and a scalpel) to dissect the fascia. More specifically, as shown in FIG. 1, the surgeon uses the forceps 10 to separate the fascia from the ulnar nerve and then retracts the forceps 10 while holding the fascia to allow for the use of scalpel 12 to cut the fascia. This often results in the surgeon repeatedly switching instruments while operating near the ulnar nerve and blood vessels.

The open procedure has several disadvantages, including the need to use both a forceps and a scalpel and to switch between the instruments several times. As a result, the surgeon has to use both hands (and may even require the assistance of a second person in some circumstances) while also increasing the risk of ulnar nerve damage (which could result in loss of motor function in the hand) and/or harm to surrounding blood vessels.

There is a need in the art for an improved device and related method for performing procedures requiring navigation of several tissue layers, such as ulnar nerve decompression.

### BRIEF SUMMARY

Discussed herein are various devices that can be used for both tissue manipulation and cutting during surgical procedures and the related methods of using such devices during such procedures.

In Example 1, a combination tissue manipulation and cutting device comprises a first elongate member comprising a first handle, a first jaw, and a first joint coupling structure disposed between the first handle and the first jaw, a second elongate member comprising a second handle, a second jaw, and a second joint coupling structure disposed between the second handle and the second jaw, a rotatable joint disposed at the first and second coupling structures such that the first and second elongate members are rotatable in relation to each other at the rotatable joint, a blade guide housing disposed along a length of the first elongate member, the blade guide housing comprising a distal opening, a proximal opening, and a lumen defined through the blade guide housing from the distal opening to the proximal opening, and a blade drive body slidably disposable within the lumen of the blade guide housing, the blade drive body comprising a blade attachment component at a distal end of the blade drive body.

Example 2 relates to the combination tissue manipulation and cutting device according to Example 1, wherein the blade drive body comprises a detent disposed on the blade drive body, wherein the detent extends beyond the cross-sectional width or height of the blade drive body such that the detent cannot fit within the lumen of the blade guide housing.

Example 3 relates to the combination tissue manipulation and cutting device according to Example 2, wherein the detent contacts a proximal end of the blade guide housing as the blade drive body is urged distally through the blade guide housing, thereby preventing the blade drive body from moving further distally through the blade guide housing.

Example 4 relates to the combination tissue manipulation and cutting device according to Example 1, wherein the blade drive body comprises at least one channel defined along the length of the blade drive body, and wherein the blade guide housing comprises at least one detent disposed on the blade guide housing such that the at least one detent is disposed within the at least one channel when the blade drive body is disposed within the blade guide housing.

Example 5 relates to the combination tissue manipulation and cutting device according to Example 1, wherein the blade drive body comprises a finger contact structure associated with a proximal portion of the blade drive body.

Example 6 relates to the combination tissue manipulation and cutting device according to Example 5, wherein the finger contact structure comprises ribs or protrusions.

Example 7 relates to the combination tissue manipulation and cutting device according to Example 1, wherein the blade guide housing and the blade drive body are removable.

Example 8 relates to the combination tissue manipulation and cutting device according to Example 1, further comprising a removable blade coupleable to the blade attachment component.

Example 9 relates to the combination tissue manipulation and cutting device according to Example 1, wherein the first elongate member comprises a guide slot defined within and disposed along a length of the first elongate member, wherein the blade drive body is slidably disposed within adjacent to or within the guide slot.

Example 10 relates to the combination tissue manipulation and cutting device according to Example 1, wherein the blade drive body is slidable between a retracted position and a deployed position along a length of the first elongate member.

In Example 11, a combination tissue manipulation and cutting device, the device comprises a first elongate member comprising a first handle, a first jaw, and a guide slot defined within and along a length of the first elongate member, a second elongate member comprising a second handle and a second jaw, wherein the second elongate member is rotatably coupled to the first elongate member at a rotatable joint, a blade guide housing disposed along a length of the first elongate member, the blade guide housing comprising a distal opening, a proximal opening, and a lumen defined through the blade guide housing from the distal opening to the proximal opening, and a blade drive body slidably disposable within the guide slot and the lumen of the blade guide housing. The blade drive body comprising a blade attachment component at a distal end of the blade drive body, and a detent disposed on the blade drive body, wherein the detent extends beyond the cross-sectional width or height of the blade drive body such that the detent cannot fit within the lumen of the blade guide housing, wherein the blade drive body is slidable between a proximal position and a distal position. The device further comprises a removable blade operably coupled to the blade attachment component.

Example 12 relates to the combination tissue manipulation and cutting device according to Example 11, wherein the detent contacts a proximal end of the blade guide housing as the blade drive body is urged distally through the blade guide housing, thereby preventing the blade drive body from moving further distally through the blade guide housing.

Example 13 relates to the combination tissue manipulation and cutting device according to Example 11, wherein the blade drive body comprises a finger contact structure associated with the blade drive body, wherein the finger contact structure comprises ribs or protrusions.

Example 14 relates to the combination tissue manipulation and cutting device according to Example 11, wherein the blade guide housing and the blade drive body are removable and replaceable.

Example 15 relates to the combination tissue manipulation and cutting device according to Example 14, further comprising at least one additional blade guide housing, wherein the at least one additional blade guide housing is interchangeable with the blade guide housing, and at least one additional blade drive body, wherein the at least one additional blade drive body is interchangeable with the blade drive body.

In Example 16, a method of manipulating and cutting tissue during a surgical procedure comprises engaging target tissue with a surgical device, the surgical device comprising a first elongate member comprising a first jaw and a blade guide housing attached to the first elongate member, a second elongate member rotatably coupled to the first elongate member, the second elongate member comprising a second jaw, and a blade drive body slidably disposed within the blade guide housing, the blade drive body being removably attached to a blade. The method further comprises urging the blade drive body and the blade distally through the blade guide housing into a deployed configuration such that the blade cuts the target tissue.

Example 17 relates to the method according to Example 16, further comprising retracting the blade drive body and the blade from the deployed configuration proximally through the blade guide housing into a retracted configuration.

Example 18 relates to the method according to Example 16, wherein the engaging the target tissue comprises grasping the tissue between the first and second jaws.

Example 19 relates to the method according to Example 16, wherein the urging the blade drive body and the blade distally comprises placing a finger on the blade drive body and urging the blade drive body distally with the finger.

Example 20 relates to the method according to Example 16, wherein the blade drive body comprises a detent, wherein the urging the blade drive body and the blade distally further comprises urging the blade drive body and the blade distally until the detent contacts a proximal end of the blade guide housing such that the blade drive body cannot be advanced distally any further.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a perspective view of a standard decompression surgery using a forceps and a scalpel.

FIG. 2A is a perspective view of a combination surgical device, according to one embodiment.

FIG. 2B is a top view of the combination surgical device of FIG. 2A, according to one embodiment.

FIG. 2C is a bottom view of the combination surgical device of FIG. 2A, according to one embodiment.

FIG. 3 is an exploded perspective view of the combination surgical device of FIG. 2A, according to one embodiment.

FIG. 4A is a perspective view of a first elongate member of a surgical device, according to one embodiment.

FIG. 4B is a top view of the first elongate member of FIG. 4A, according to one embodiment.

FIG. 4C is a close-up perspective view of a portion of the first elongate member of FIG. 4A, according to one embodiment.

FIG. 5A is a perspective view of a first elongate member and a blade guide structure of a surgical device, according to one embodiment.

FIG. 5B is a perspective view of the blade guide structure of FIG. 5A, according to one embodiment.

FIG. 6A is a perspective view of a slidable drive body of a surgical device, according to one embodiment.

FIG. 6B is a perspective view of another slidable drive body of a surgical device, according to another embodiment.

FIG. 7 is a perspective view of a second elongate member of a surgical device, according to one embodiment.

FIG. 8A is a perspective view of a combination surgical device in which the slidable drive body and the blade are in the deployed or cutting position, according to one embodiment.

FIG. 8B is another perspective view (from the opposite side) of the combination surgical device of FIG. 8A in the deployed or cutting position, according to one embodiment.

FIG. 9A is a perspective view of the combination surgical device of FIG. 8A in which the slidable drive body and the blade are in the retracted or undeployed position, according to one embodiment.

FIG. 9B is another perspective view (from the opposite side) of the combination surgical device of FIG. 8A in the retracted or undeployed position, according to one embodiment.

FIG. 10A is a perspective view of another combination surgical device, according to an alternative embodiment.

FIG. 10B is a perspective view of the combination surgical device of FIG. 10A with a slidable drive body and blade included, according to an alternative embodiment.

FIG. 11A is a perspective view of a first elongate member and blade guide structure of a combination surgical device, according to another embodiment.

FIG. 11B is another perspective view of the first elongate member and blade guide structure of FIG. 11A with one side of the guide structure removed, according to one embodiment.

FIG. 11C is an expanded perspective view of the wall of the blade guide structure of FIG. 11B, according to one embodiment.

FIG. 12 is a perspective view of a slidable blade drive body, according to an alternative embodiment.

FIG. 13A is a perspective view of a combination surgical device in which the slidable drive body is in the retracted or undeployed position, according to one embodiment.

FIG. 13B is a perspective view of the combination surgical device of FIG. 13A in which the slidable drive body is in the deployed or cutting position, according to one embodiment.

FIG. 14A is a perspective view of a first elongate member and an alternative blade guide structure of a surgical device, according to a further embodiment.

FIG. 14B is a perspective view of the blade guide structure of FIG. 14A, according to one embodiment.

FIG. 14C is a top perspective view of the blade guide structure of FIG. 14A, according to one embodiment.

FIG. 14D is a top view of the flat sheet of metal prior to being formed into the blade guide structure of FIG. 14A, according to one embodiment.

FIG. 15A is a perspective view of a method of surgery using a combination device in which the jaws are disposed under the fascia layer, according to one embodiment.

FIG. 15B is another perspective view of the combination device of FIG. 15A in which the jaws are widened, according to one embodiment.

FIG. 15C is another perspective view of the combination device of FIG. 15A in which the blade drive body and blade are urged distally into contact with the fascia layer, according to one embodiment.

FIG. 15D is another perspective view of the combination device of FIG. 15A in which the blade drive body and blade are urged further distally such that the fascia layer is severed, according to one embodiment.

FIGS. 16A, 16B, and 16C are graphs depicting the results of a force test as described in the Example, according to one embodiment.

FIGS. 17A, 17B, and 17C are graphs depicting the results of the “number of cuts” test as described in the Example, according to one embodiment.

FIGS. 18A, 18B, and 18C are graphs depicting the results of the “percentage cut” test as described in the Example, according to one embodiment.

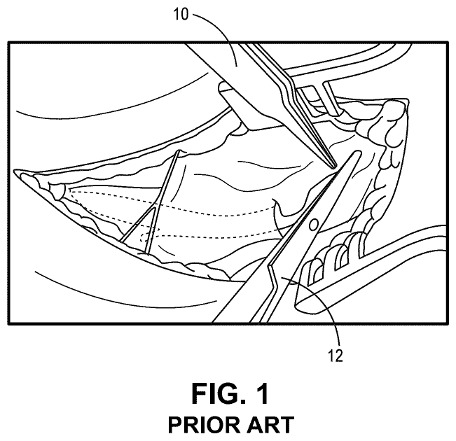

### DETAILED DESCRIPTION

The various embodiments herein relate to a surgical device that operates as both a forceps and a scalpel and the related methods for using the device. According to certain implementations, such a device can reduce the number of instruments required for certain procedures and thus minimize the risk of damage to the surrounding structures. The surgical device could be used for a variety of procedures, including, for example, ulnar nerve release or decompression surgery, carpal tunnel surgery, and fasciotomies.

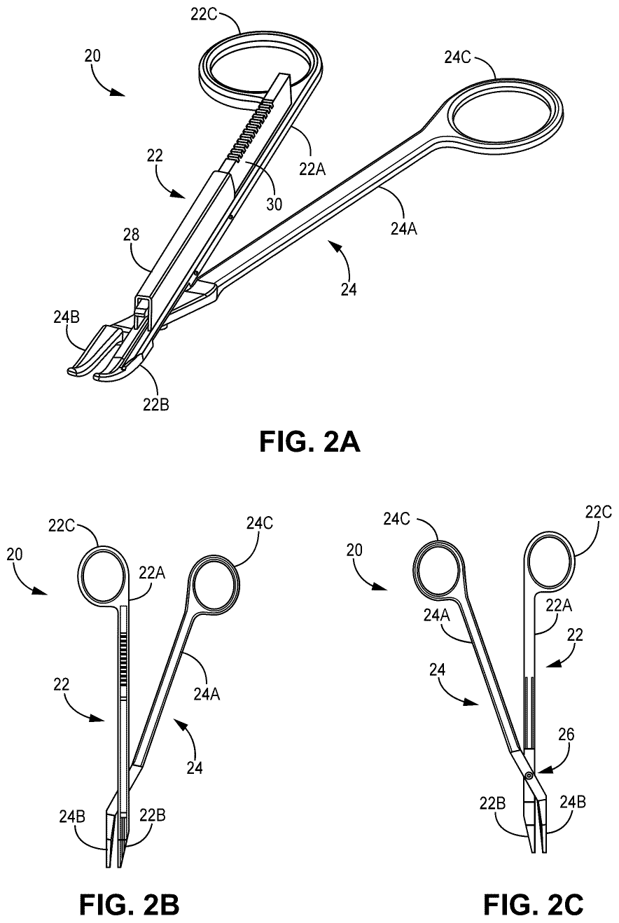

One exemplary embodiment of a surgical device 20 is depicted in FIGS. 2A-2C. The device 20 has a pair of elongate members 22, 24 that are coupled together at a rotatable joint 26 (as best shown in FIG. 2C) such that the members 22, 24 can rotate around the joint 26 in relation to each other. Each member 22, 24 has a handle section 22A, 24A disposed proximally of the joint 26 and a jaw section 22B, 24B disposed distally of the joint 26. Further, in this particular embodiment, each of the handles 22A, 24A also has a finger loop 22C, 24C to facilitate the surgeon grasping the device 20. Alternative device 10 embodiments have no finger loops. In addition, the device 20 has a blade guide structure 28 attached to the first elongate member 22 and a slidable blade drive body 30 that can be slidably disposed through the blade guide structure 28.

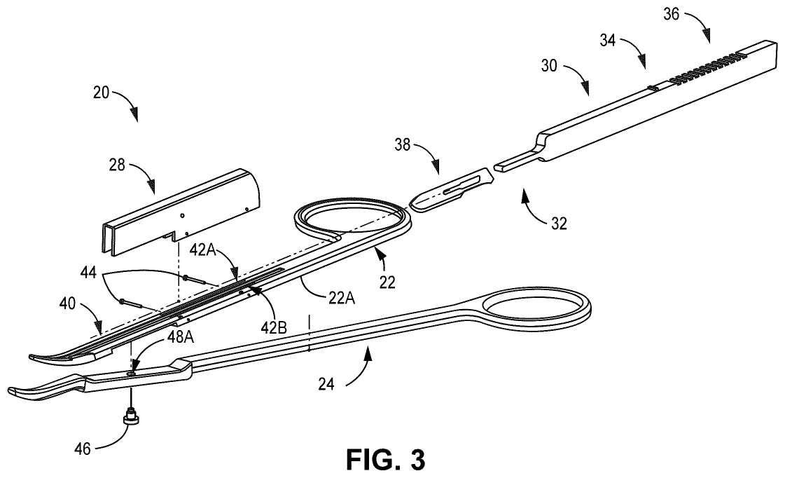

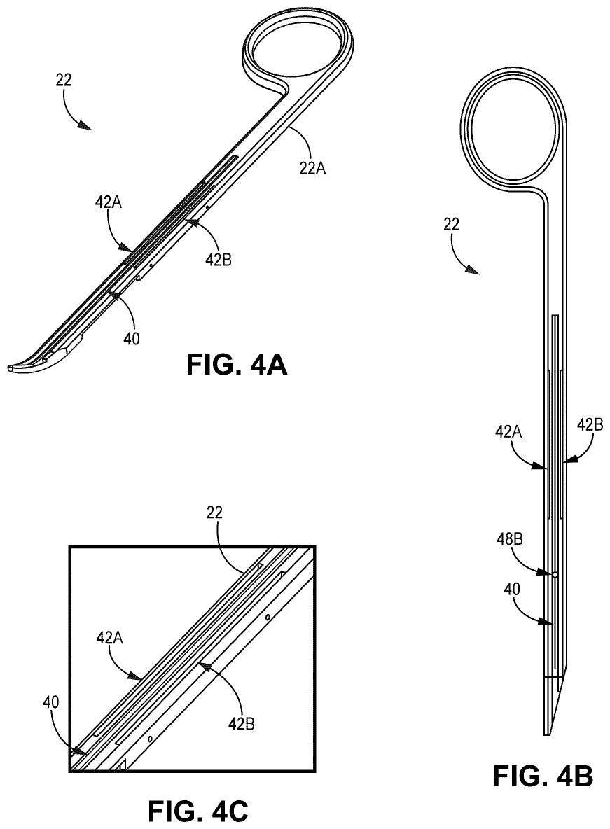

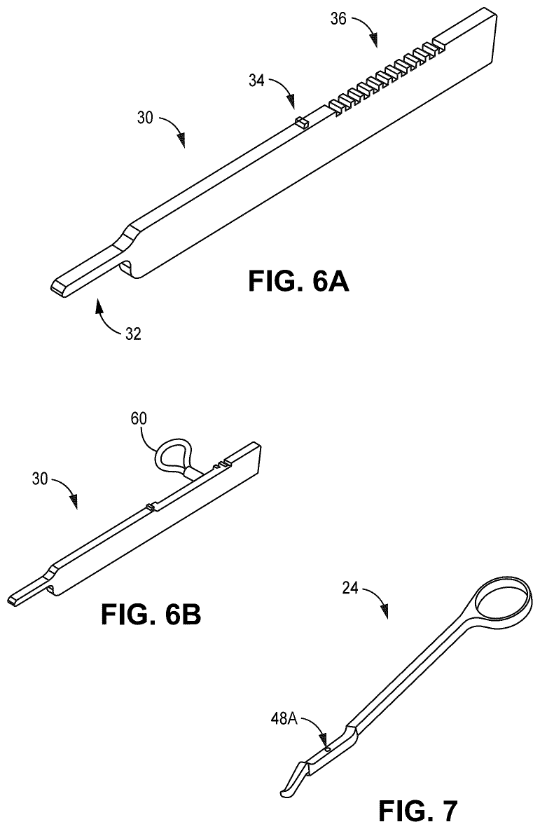

In one specific implementation as shown in FIG. 3, the blade guide structure 28 is a housing 28 that is attached to the first elongate member 22 via the two attachment slots 42A, 42B defined in the handle 22A (as also shown in FIGS. 4A-C). Further, as shown in both FIG. 3 and FIG. 6, the slidable drive body 30 has a blade attachment component 32 at its distal end such that the blade 38 can couple to the attachment component 32. The drive body 30 in this embodiment also has a detent 34 disposed on the drive body 30 that can prevents distal advancement of the drive body 30 past the point at which the detent 34 contacts the proximal end of the guide structure 28. In addition, in certain embodiments, the first elongate member 22 has a guide slot 40 (also shown in FIGS. 4A-4C) defined along the length of the member 22 such that a bottom portion of the blade 38 or the drive body 30 can be disposed within the slot 40. As a result, the sliding motion of the blade 38 and drive body 30 along the first elongate member 22 and through the guide structure 28 can be maintained along a predetermined path established by the slot 40.

Further, in accordance with certain embodiments, the drive body 30 and the slot 40 prevent the dulling of the blade 38. That is, the attachment of the blade 38 to the drive body 30 and the positioning of the drive body 30 and the blade 38 in the slot 40 ensures that the blade 38 does not make contact with the elongate member 22 as the drive body 30 and blade 38 are slid along the elongate member 22 during use, thereby minimizing or preventing the dulling of the blade as a result of contact with the elongate member 22 during use.

Any of the device embodiments disclosed or contemplated herein (including device 20 and device 100 discussed further below) can use any standard scalpel blade. For example, in one specific implementation, the blade 38 discussed above (and any blade used with any implementation herein) can be a Bard-Parker™ number 10 scalpel blade commercially available from Aspen Surgical™ (https://www.aspensurgical.com/products/surgical-essentials/blades-scalpels/conventional-blades/). Alternatively, the various blade drive body embodiments disclosed or contemplated herein (such as body 30 or body 110) and device implementations can be modified such that various known scalpel blade for use in surgical scalpels can be used.

Known attachment components and mechanisms can be used to attach the various components of the device 20 thereto. For example, screws or bolts 44 as shown in FIG. 3 can be used to attach the guide structure 28 to the first elongate member 22. Alternatively, an adhesive such as one of the adhesives commercially available from Loctite® (or any other known adhesive that can be used to attach to components of a medical device) can be used to attached the guide structure 28 and the first elongate member 22. In a further alternative, any other similar attachment components or methods can be used. Further, a shoulder screw 46 is coupled to the first and second members 22, 24 at the joint 16 to allow for rotation of the two members 22, 24 at the joint 16. In one specific embodiment, the shoulder screw 46 has a length such that it extends through the second elongate member 24 via opening 48A (as shown in FIGS. 3 and 7) and into the first elongate member 22 via opening 48B (as shown in FIG. 4B). However, the screw 46 does not extend so far through the opening 48B that it extends into the slot 40 defined in the first elongate member 22, thereby ensuring that the distal end of the screw 46 does not block the sliding path of the blade 38 and drive body 30 as described above. Further, one of ordinary skill understands that any attachment components, mechanisms, or features can be used in the device 20.

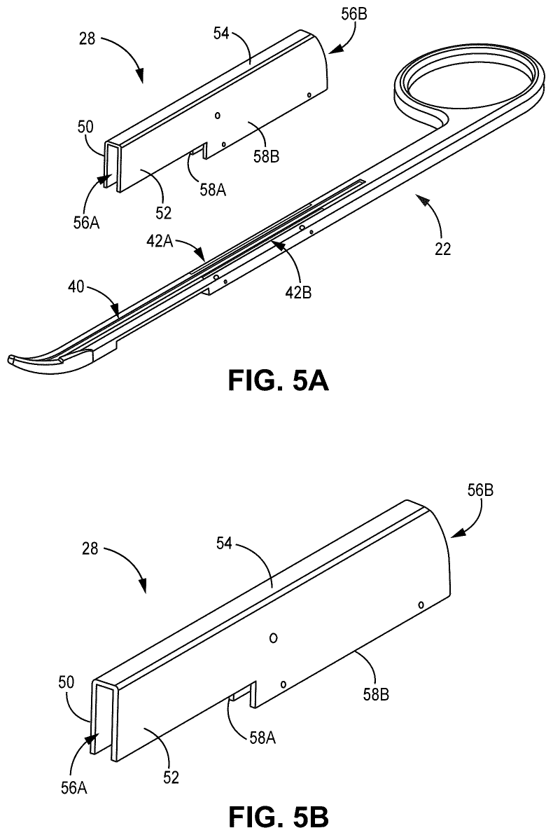

One exemplary implementation of the blade guide structure 28 is shown in additional detail in FIGS. 5A and 5B. The structure 28 in this embodiment is a housing 28 having a first side 50, a second side 52, a top cover 54, and distal and proximal openings 56A, 56B defined in each end of the housing 28 as shown. In addition, the housing 28 has two projections 58A, 58B extending from the two sides 50, 52 as shown that can be disposed within the attachment slots 42A, 42B discussed above. Alternatively, the blade guide structure 28 can be attached to the first member 22 via any known attachment component or mechanism.

A slidable drive body 30 in accordance with one specific embodiment is shown in FIG. 6A. As mentioned above, the drive body 30 has a blade attachment component 32 at its distal end, along with a detent 34 disposed on the drive body 30 that can prevents distal advancement of the drive body 30 past the point at which the detent 34 contacts the proximal end of the guide structure 28. The detent 34 can be a protrusion 34 as shown. Alternatively, the detent 34 can be any structural component or feature disposed, defined, or attached anywhere on the drive body 30 that can prevent the further distal advancement of the drive body 30 through the guide structure 28 at a specific, predetermined point. In this specific implementation, the detent 34 is a protrusion 34 disposed on the top edge of the body 30 such that, as the drive body 30 is urged distally through the guide structure 28, the protrusion 34 will advance into contact with the top cover 54 of the guide structure 28 such that the protrusion 34 can advance no further, thereby preventing further distal advancement of the drive body 30 through the housing 28. In addition, in certain embodiments, the drive body 30 also has a finger contact section 36 disposed along the top edge of the drive body 30 proximal of the detent 34. In the specific implementation as shown, the finger contact section 36 is a set of ribs 36 formed in the top edge of the body 30 such that the surgeon or other user can easily establish frictional contact when the surgeon places her finger(s) on the top of the drive body 30 to urge it distally or proximally during use. Alternatively, the finger contact section 36 can be any other surface feature, structural component, or structural feature that facilitates the use of a finger to slide the drive body 30 during use as described herein. For example, in an alternative embodiment as shown in FIG. 6A includes a finger ring 60 that the surgeon or other user can use to urge the drive body 30 distally and proximally as described herein.

FIG. 7 depicts an exemplary embodiment of the second elongate member 24. The member 24 shows the opening 48A defined therethrough as discussed above such that the rotatable attachment mechanism (such as the shoulder screw 46 discussed above) can be positioned therethrough to form the joint 26. Alternatively, the second member 24 can be rotatably coupled to the first member 22 via any known component or mechanism.

According to certain implementations, the blade drive body 30, the blade 38, and the blade guide housing 28 are all modular. That is, each of the drive body 30, the blade 38, and the housing 28 is removable and replaceable and can be available in multiple different types and sizes for attachment to the same first elongate member 22 to accommodate different blade types and sizes and/or to accommodate different surgeon needs. Thus, in various embodiments, any device embodiment herein can come as a set of modular, interchangeable components that includes two or more different, interchangeable drive bodies 30 and two or more different, interchangeable housings 28 (and thus allows for use with two or more different types of blades 38—which may be provided with the set or separately—and/or to accommodate different surgeon needs).

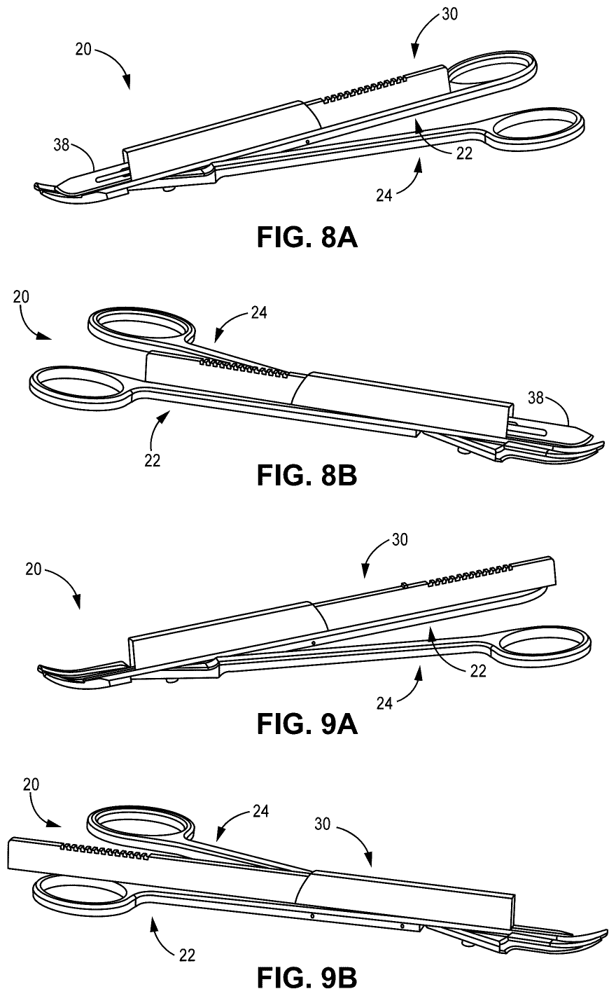

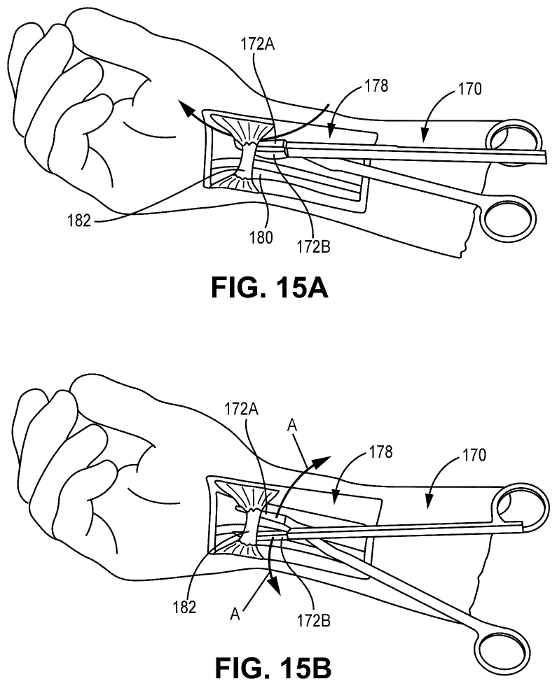

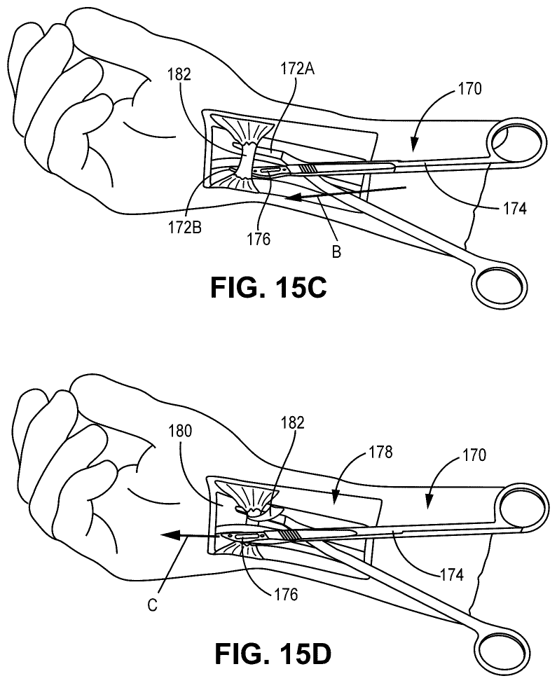

In use, a surgeon or other medical professional can use the device 20 to perform both tissue manipulation (distraction, grasping, etc.) and cutting actions. As best shown in FIGS. 8A and 8B, when the user wants to perform a cutting action, the user can urge the drive body 30 (and thus the blade 38) distally into the deployed position. Further, when the cutting action is completed or when the device 20 is not in use, the drive body 30 (and thus the blade 38) can be urged proximally into is retracted position, as shown in FIGS. 9A and 9B. Additional detail about an exemplary procedure is described below with reference to FIGS. 15A-15D.

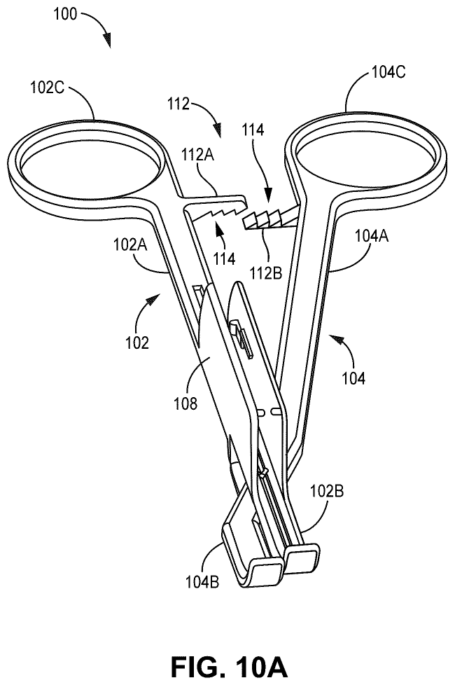

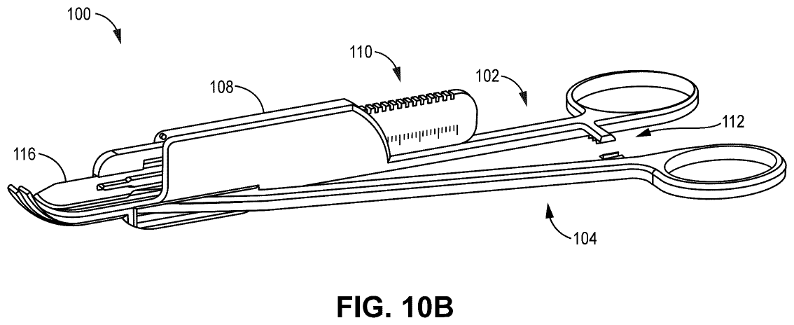

An alternative embodiment of a surgical device 100 is depicted in FIGS. 10A and 10B. The components, features, attachment mechanisms, and functionality of this device 100 are the same or substantially similar to the corresponding component, features, and functionality of the surgical device 20 discussed above, except as expressly discussed below. The device 100 has a pair of elongate members 102, 104 that are coupled together at a rotatable joint 106 such that the members 102, 104 can rotate around the joint 106 in relation to each other. Each member 102, 104 has a handle section 102A, 104A disposed proximally of the joint 106 and a jaw section 102B, 104B disposed distally of the joint 106. Further, in this particular embodiment, each of the handles 102A, 104A also has a finger loop 102C, 104C to facilitate the surgeon grasping the device 100. Alternative device embodiments have no finger loops. In addition, the device 100 has a blade guide structure 108 attached to the first elongate member 102 that is configured to slidably receive a slidable blade drive body 110 with a blade 116 attached thereto (as best shown in FIG. 10B). Known attachment components and mechanisms can be used to attach the various components of the device 100 thereto.

The exemplary implementation of the device 100 as shown also has a retention or locking mechanism 112 that can be used to hold the device 100 in the clamped position when desired. More specifically, the locking mechanism 112 as shown is a ratchet mechanism 112 having two arms 112A, 112B with structural ratcheting features 114 (such as engageable ribs or the like) defined or formed on the opposing sides of the arms 112A, 112B as shown such that the two arms 112A, 112B can engage with each other (via the ratcheting features 114) to lock the two handles 102A, 102B in place, thereby locking the jaws 102B, 104B in place as desired during a procedure. Alternatively, any known locking mechanism can be used.

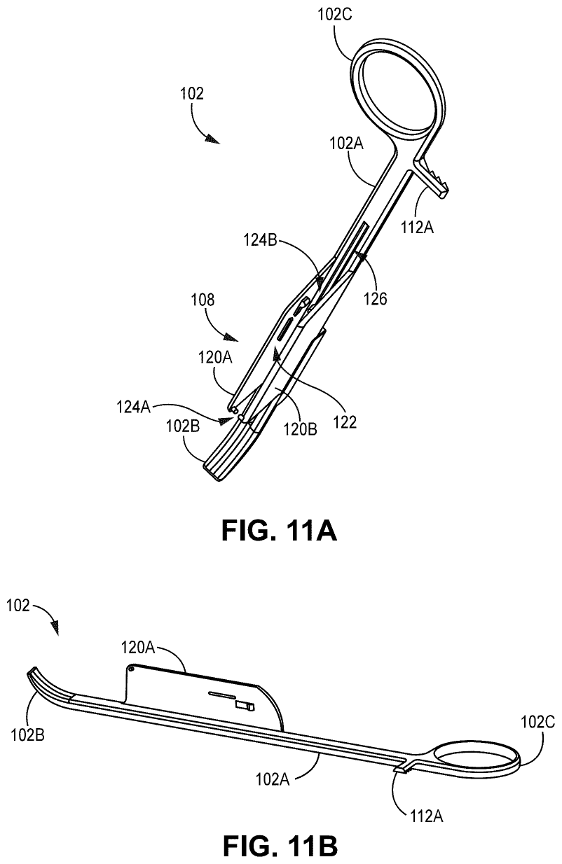

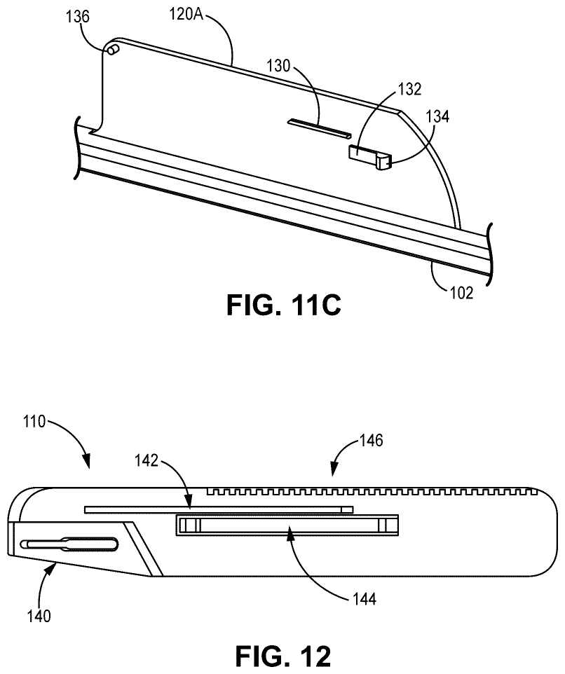

One exemplary implementation of the first elongate member 102 and the blade guide structure 108 disposed thereon are shown in additional detail in FIGS. 11A-11C. The structure 108 in this embodiment is a housing 108 having a first side wall 120A, a second side wall 120B, a top opening 122 defined along the length of the housing 108 by the two side walls 120A, 120B, and distal and proximal openings 124A, 124B defined in each end of the housing 108 as shown. In certain embodiments, the two side walls 120A, 120B are integral with the first elongate member 102 as a result of various manufacturing processes such as molding or 3D printing for example. Alternatively, the two sides 120A, 120B can be attached to the first elongate member 102 via any known attachment process (such as welding or the like), component, or mechanism. In addition, in certain embodiments, the first elongate member 102 has a guide slot 126 (also shown in FIG. 11A) defined along the length of the member 102 such that a bottom portion of the blade 116 or the drive body 110 can be disposed within the slot 126. As a result, the sliding motion of the blade 116 and drive body 110 along the first elongate member 102 and through the guide structure 108 can be maintained along a predetermined path established by the slot 126. Alternatively, certain embodiments of the first elongate member 102 has no guide slot.

FIG. 11C depicts the first side 120A of the blade guide structure 108 in accordance with one embodiment. It is noted that both sides 120A, 120B have identical or substantially similar components or features such that the second side 120B (not shown in FIG. 11C) can essentially be a mirror image of the first side 120A as shown. In other words, the components and/or features of the first side 120A as shown in FIG. 11C and discussed below are also present on the second side 120B, according to one embodiment. The inner wall of the first side 120A has a first guide rail 130 and a second guide rail 132 with a detent 134 at one end of the rail 132. As will be described in additional detail below, both rails 130, 132 and the detent 134 are configured to be coupleable with the corresponding guide grooves 142, 144 defined in the drive body 110 such that the coupling features help to maintain the positioning of the drive body 110 as it is urged distally and proximally through the guide structure 108 during use. Further, the inner wall of the first side 120A also have a protrusion 136 near the distal end of the side 120A that can help to retain the drive body 110 within the guide structure 108 during use such that the drive body 110 does not pass through the top opening 122. Again, as mentioned above, the second side 120B can also have the same guide rails, detent, and protrusion (not shown). Alternatively, the two sides 120A, 120B can have any known mechanisms, structures, or features for providing stability and retention to the drive body 110 while the drive body 110 is moved distally and proximally through the housing 108.

A slidable drive body 110 in accordance with one specific embodiment is shown in FIG. 12. The drive body 110 has a blade attachment component 140 at its distal end, a first guide groove 142 defined on both sides of the body 110 and a second guide groove 144 also defined on both sides of the body 110 as shown. The first guide groove 142 is positioned such that the first guide rails of the housing 108 (including first guide rail 130 of the first side wall 120A as discussed above) can be slidably disposed therein. Similarly, the second guide groove 144 is positioned such that the second guide rails of the housing 108 (including the second guide rail 132 and the detent 134 of the first side wall 120A as discussed above) can be slidably disposed therein.

The detent 134 on the first side wall 120A (and the corresponding detent on the second side wall 120B) can be a protrusion 134 as shown. Alternatively, the detent 134 can be any structural component or feature disposed, defined, or attached on the first side wall 120A and disposed within the second guide groove 144 such that the detent 134 prevents the further advancement of the drive body 110 distally or proximally through the guide structure 108 past a certain, predetermined point. In this specific implementation, as the drive body 110 is urged distally through the guide structure 108, the proximal end of the second guide groove 144 will advance until it makes contact with the protrusion 134 such that the drive body 110 can advance no further, thereby preventing further distal advancement of the drive body 110 through the housing 108. Similarly, as the drive body 110 is urged proximally through the guide structure 108, the distal end of the second guide groove 144 will advance until it makes contact with the protrusion 134 such that the drive body 110 can advance no further, thereby preventing further proximal advancement of the drive body 110 through the housing 108.

In addition, in certain embodiments, the drive body 110 also has a finger contact section 146 disposed along the top edge of the drive body 110. In the specific implementation as shown, the finger contact section 146 is a set of ribs 146 formed in the top edge of the body 110 such that the surgeon or other user can easily establish frictional contact when the surgeon places her finger(s) on the top of the drive body 110 to urge it distally or proximally during use. Alternatively, the finger contact section 146 can be any structural component or feature that facilitates the use of a finger to slide the drive body 110 during use as described herein. For example, like the alternative embodiment as shown in FIG. 6A, the drive body 110 can have a finger ring (not shown) attached thereto that the surgeon or other user can use to urge the drive body 110 distally and proximally as described herein.

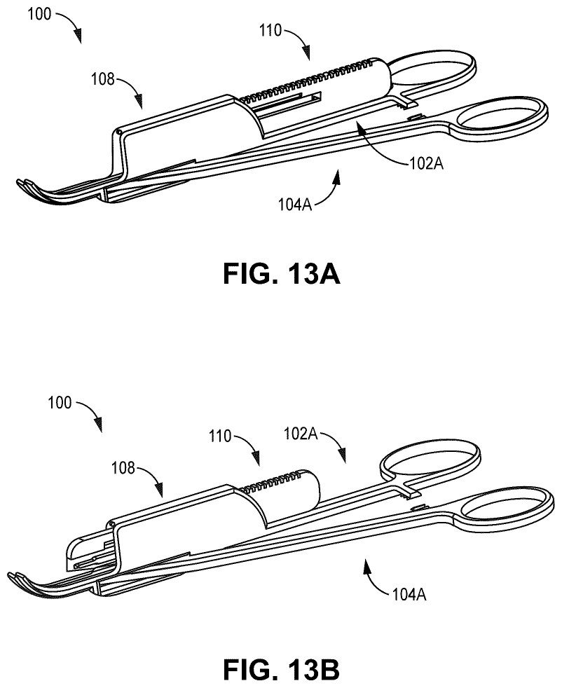

In use, a surgeon or other medical professional can use the device 100 to perform both tissue manipulation (distraction, grasping, etc.) and cutting actions in a fashion similar to that described with respect to device 20. As best shown in FIG. 13A, when the cutting action is completed or when the device 100 is not in use, the drive body 110 (and thus the blade such as blade 116 as shown in FIG. 10B) can be urged proximally into its retracted position. In contrast, as shown in FIG. 13B, when the user wants to perform a cutting action, the user can urge the drive body 110 (and thus the blade such as blade 116 as shown in FIG. 10B) distally into the deployed position.

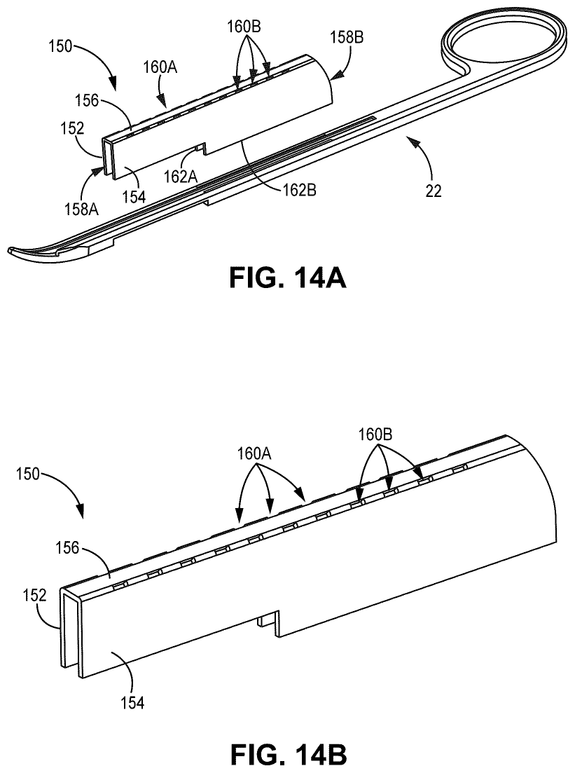

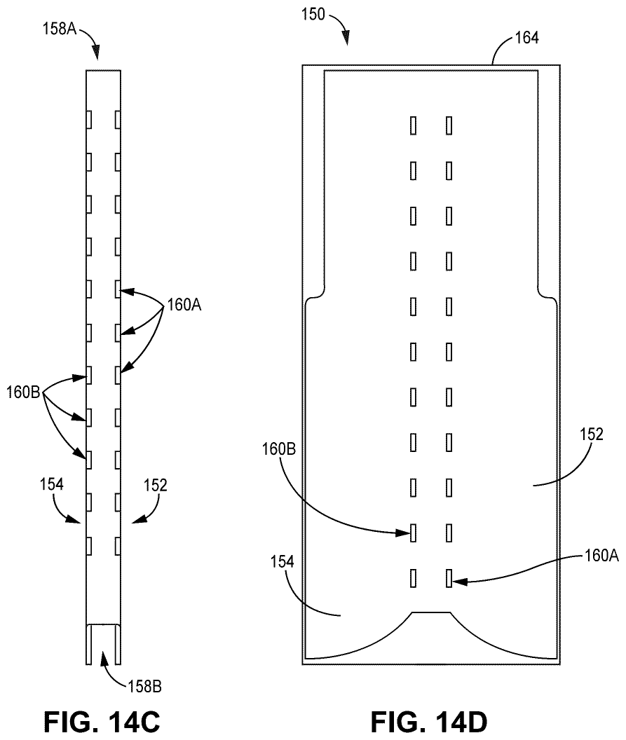

Another embodiment of a blade guide structure 150 is shown in additional detail in FIGS. 14A-14D. The components, features, attachment mechanisms, and functionality of this housing 150 are the same or substantially similar to the corresponding component, features, and functionality of the blade guide housing 28 discussed above, except as expressly discussed below. The structure 150 in this embodiment is a housing 150 having a first side 152, a second side 154, a top cover 156, distal and proximal openings 158A, 158B defined in each end of the housing 150, and two sets of openings 160A, 160B defined along the length of the housing 150 as shown. As best shown in FIGS. 14B and 14C, the first set of openings 160A is defined along the edge between the first side 152 and the top cover 156, and the second set of openings 160B is defined along the edge between the second side 154 and the top cover 156. The housing 150 also has two projections 162A, 162B extending from the two sides 152, 154 as shown that can be disposed within the attachment slots 42A, 42B discussed above. Alternatively, the blade guide structure 150 can be attached to the first member 22 via any known attachment component or mechanism.

In accordance with certain implementations, the two sets of openings 160A, 160B are incorporated into the housing 150 in order to facilitate the formation or manufacturing of the housing 150. More specifically, as best shown in FIG. 14D, the housing 150 can be constructed by starting with a flat piece of metal 164 that is formed into the desired shape of the housing 150 by simply bending portions of the flat sheet 164 at the openings 160A, 160B. More specifically, the flat sheet 164 is formed with the two sets of openings 160A, 160B disposed adjacent to and parallel with each other along the length of the sheet 164. As such, the portion of the sheet 164 that will become the first side 152 can be bent in relation to the rest of the sheet 164 along the length of the openings 160A such that the openings 160A are disposed along the edge between the first side 152 and the top cover 156, while the portion of the sheet 164 that will become the second side 154 can be bent in relation to the rest of the sheet 164 along the length of the openings 160B such that the openings 160B are disposed along the edge between the second side 154 and the top cover 156. In other words, the openings 160A, 160B are disposed on the sheet 164 to facilitate the bending of the sheet 164 as a result of each of the line of openings 160A, 160B creating a predetermined line along with the sheet 164 is “weaker” or more susceptible to bending. As a result, the sheet 164 can be bent along each line of openings 160A, 160B as described above to form the final shape of the housing 150 as shown in FIGS. 14A-14C. The resulting housing 150, according to various embodiments, has smooth interior and exterior surfaces that have no grooves or protrusions. As a result, the housing 150 is easy to manufacture inexpensively while maintaining structural integrity and functionality in comparison to other versions.

One exemplary method of using any of the device embodiments disclosed or contemplated herein is depicted in additional detail in FIGS. 15A-15D, according to one implementation. In the method, once the appropriate incision 178 is made at the target area (in this case, the wrist), a version of the device 170 is positioned within the incision 178 such that the jaws 172A, 172B are disposed superior to the nerve layer 180 and inferior to the fascia layer 182, as best shown in FIG. 15A. Further, as shown in FIG. 15B, once the jaws 172A, 172B are so positioned, the jaws 172A, 172B are widened as shown via arrows A to prepare the device 170 and the target fascia layer 182 for cutting the layer 182. At this point, as depicted in FIG. 15C, the blade drive body 174 and the attached blade 176 are urged distally as shown via arrow B such that the blade 176 makes contact with the target fascia layer 182. Further, as shown in FIG. 15D, the drive body 174 and blade 176 continue to be urged distally as shown via arrow C until the blade 176 cuts through and severs the fascia layer 182 as shown without damaging the underlying layer of nerves 180.

During use, one of the additional benefits of the slot in the various implementations herein (such as slot 40 or slot 126, for example) is that the slot can facilitate the cutting of tissue. More specifically, the positioning of the blade (such as blade 38, 116, or 176) within the slot allows for the blade to still cut through the tissue regardless of slight deflection of the blade due to tissue sitting atop the jaw (such as jaw 172B in FIGS. 15A-15D). That is, because the bottom portion of the blade is disposed within the slot, the lowest point of the blade is disposed at a position that is lower than the bottom of the tissue such that the blade can cut through the tissue without issue, even if the blade is urged or “deflected” slightly upward as a result of the presence of the tissue.

Of course, those of ordinary skill in the art understand that the method as described above and depicted in FIGS. 15A-15D is only one exemplary procedure, and that the various device embodiments herein can be used in a variety of different known procedures that require manipulation and cutting of target tissue.

The various device embodiments and related methods disclosed or contemplated herein (including devices 20 and 100, and optional components thereof such as housing 150, as discussed in detail above) provide advantages over the known technologies. That is, the device and method implementations herein minimize the number of tools or devices required for manipulating and cutting tissue, thereby minimizing the risk of injury to the patient. Further, the device implementations herein include a fully enclosed retracted position for the blade in which the blade is fully housed within the blade guide structure, thereby reducing the risk of inadvertent exposure of the blade to the patient and surgeon. Additionally, the movement of the blade in each embodiment herein is restricted to one-dimensional movement as it is guided along a single path between its retracted position and deployed position by the blade guide housing and other structures and features as described herein.

### EXAMPLE

### Summary

This study quantified the cutting ability of one embodiment of the unique device as described herein as compared to its gold standard counterparts in the laboratory. This force comparison was meant to demonstrate similar cutting ability to known devices and techniques with a unique device that limits iatrogenic injuries.

Comparative biomechanical testing was performed with one embodiment of the novel device, iris scissors, bandage scissors, and a scalpel on an MTS Static Materials Test System. The peak force to slide-cut, number of cut attempts, and percentage cut on first attempt were compared between the prototype and traditional surgical tools. The materials cut in testing were Ace™ bandage, stockinette, and gauze. Statistical analyses were performed using Welch's t-tests and Fisher's exact tests.

As will be explained in additional detail below, the results showed that compared to conventional bandage and iris scissors, the novel surgical instrument required significantly less force to cut through an Ace™ bandage, stockinette, and gauze (p<0.01). The number of cuts required to transect those same materials with the novel device was comparable to that of the scalpel and bandage scissors. Additionally, while there were no differences between the novel device and the other devices for an Ace™ bandage and stockinette, the novel device tended to cut a greater percentage of gauze in one pass than did the iris scissors.

Thus, as will be discussed in further detail below, the novel surgical instrument required less force compared to conventional scissors, demonstrated cutting efficiency similar to that of a scalpel blade, and had more safety features than either instrument. This example highlights that the various embodiments herein have an improved design and functionality that have the potential to reduce iatrogenic injuries.

### Methods

For purposes of the biomechanical testing, four instruments were tested, including a prototype of the novel instrument, a #10 scalpel (Southmedic Inc., Barrie, Ontario, Canada), bandage scissors (Medline Industries, Northfield, IL), and iris scissors (Medline Industries, Northfield, IL). Medical supplies of varying elasticity and strength were selected as testing media, including gauze (Medline Industries, Northfield, IL), stockinette (Tetra Medical Supply Corp. Niles, IL), and Ace™ bandage (Medline Industries, Northfield, IL).

Each material was affixed to a uniaxial testing machine (MTS Criterion C43) between two tension grips (MTS, Advantage Screw Action 2000 Grips) connected to a 1 kN load cell (MTS, LPS. 103) and preloaded to 19.58+/−0.65 N prior to each trial. Three variables were measured: peak force to slide-cut by pushing with an open static tine(s), number of cut attempts to completely section the material, and percentage cut on first attempt. Five trials were performed for each combination of cutting device and material, with a new instrument or blade used for each. The peak force was defined as the difference between the maximum force measured during the trial and the minimum force measured prior to reaching the maximum force. The percentage of cut completion was measured as the length cut by the device divided by the total length of cut necessary to separate the material into two pieces. The values for percentage cut were evaluated with the following ranges: 0-24%, 25-49%, 50-74%, 75-99%, and 100% cut.

### Statistical Analysis

The small sample sizes (5 trials for each device on each material) were considered when selecting applicable statistical tests. Thus, for the Force and Number of Cuts tests, Welch's t-tests were used to analyze the performance of the novel device compared to the scalpel, iris and bandage scissors. Since the data for the Percentage Cut test were categorical, Fisher's exact tests were performed using an online statistical calculator. MATLAB (R2022b) was used for the Welch's t-tests and for graphical presentation.

### Results

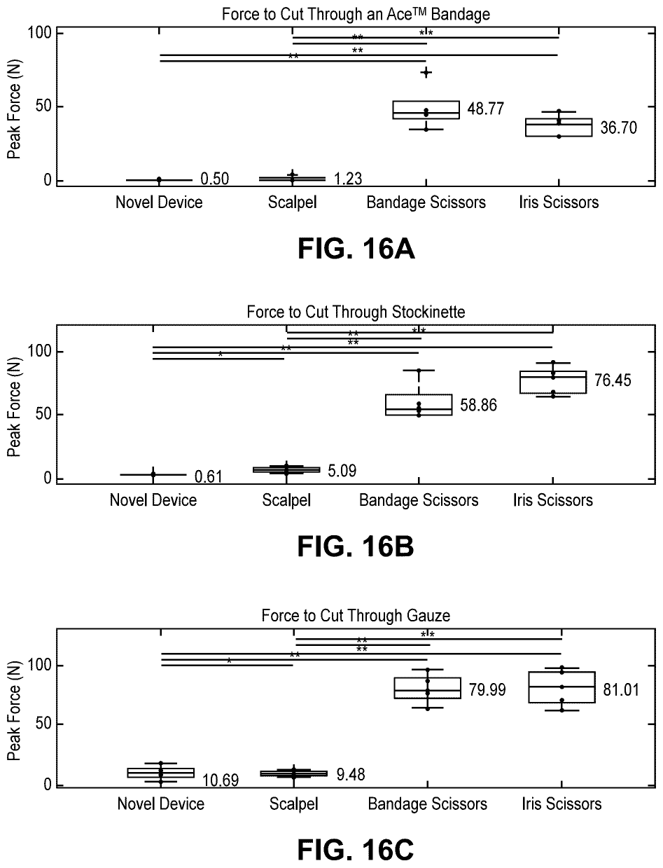

In the force test, the novel device prototype in this example required significantly less force to cut through all materials than both bandage scissors and iris scissors (p<0.01, for each combination). Compared to the scalpel for cutting stockinette, the prototype required significantly less force (p<0.05), but not compared to the scalpel for cutting gauze and the Ace™ bandage. For all three materials, the scalpel also required significantly less force than did the iris and bandage scissors (p<0.01), but there was no statistically significant difference between the bandage and iris scissors. The results are shown in FIGS. 16A-16C.

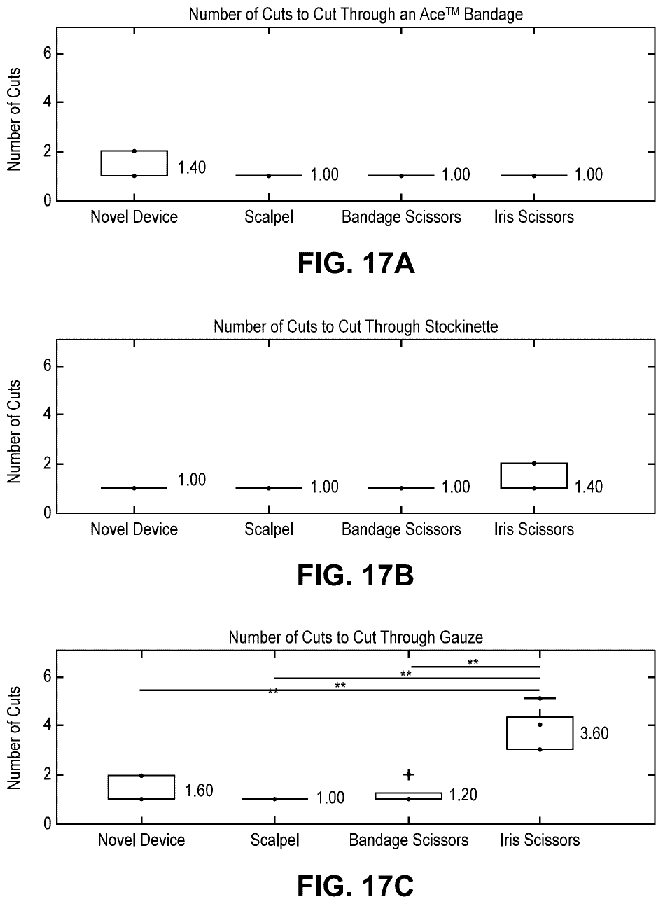

In the “number of cuts” test, the #10 scalpel blade required the fewest cuts, consistently transecting each material by applying only one stroke. The bandage scissors averaged 1.0 cut for the stockinette and Ace™ bandage, and 1.2 cuts for the gauze. The novel instrument prototype averaged 1.0 cut for the stockinette, 1.4 for the Ace™ bandage, and 1.6 for the gauze. The iris scissors required 1.4 cuts for the stockinette and 1.0 cut for the Ace™ bandage. For gauze, 3.6 attempts were required for the iris scissors, which was significantly greater than the cuts using the other instruments (p<0.01). FIGS. 17A-17C show the results of these tests.

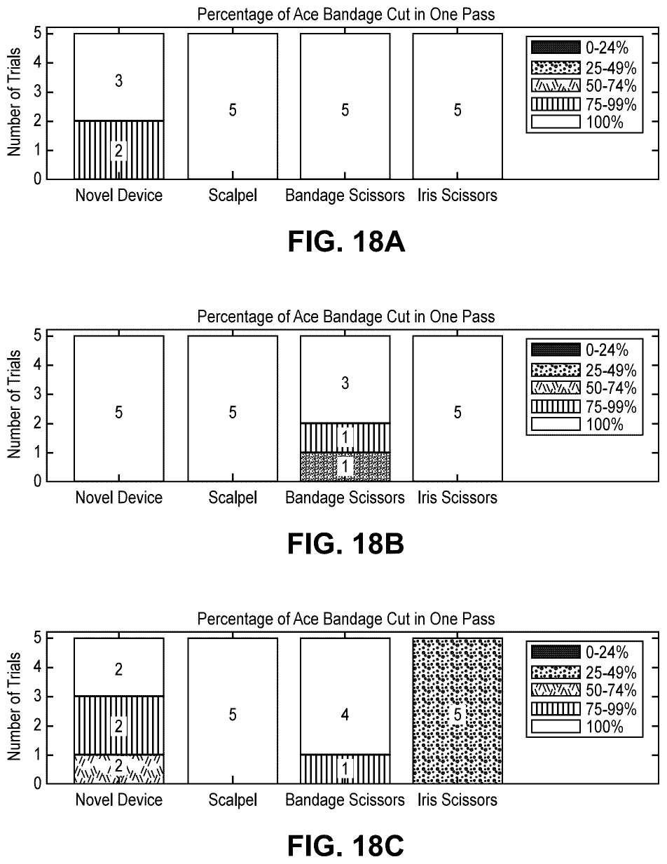

In the percentage cut test, the scalpel, bandage scissors, and iris scissors were able to cut through the Ace™ bandage in one pass for every trial. The novel device prototype cut through the Ace™ bandage in one pass, except for two instances where it cut through 75-99% of the Ace™ bandage in one pass. The scalpel, bandage scissors, and prototype cut through the stockinette in one pass for every trial. The iris scissors cut through the stockinette in one pass except for two instances, where the first pass cut through 25-50% and 75-99% of the stockinette one time each. The scalpel cut through the gauze in one pass every time. When using the bandage scissors, the gauze was cut in one pass every time except for one instance where the first cut was 75-99%. The novel device cut through the gauze completely in two instances, 75-99% in another two instances, and 50-75% in one instance. The iris scissors cut through 25-50% of the gauze for all trials. FIGS. 18A-18C each show a graphical representation of the results of this test.

### Discussion

Iatrogenic injuries to peripheral neurovascular structures continue to occur while surgery is performed on the appendicular skeleton. Although surgeons are familiar with the current instrumentation and its technical use, there are few built-in safeguards available to prevent injury. The primary aim of this example was to clearly delineate the fabrication process from idea conception to a fully functional surgical instrument and evaluate its cutting ability against established benchmarks in the field. The creation of the novel surgical device embodiments as described herein was driven by the imperative to enhance the safety profile of existing open surgical instruments, while ensuring an intuitive design that seamlessly integrates with modern techniques. The functional performance of the described novel device prototype matches that of a scalpel blade when assessing peak cutting force with a design that limits iatrogenic injury. Furthermore, significantly less force is needed to transect the Ace™ bandage, stockinette, or gauze in comparison to both iris and bandage scissors.

The novel device embodiments herein were designed to serve as a hybrid instrument, functioning similarly to a tonsil for blunt dissection, while also incorporating the capabilities of a scalpel, resulting in a protected, sharp transection. By incorporating a slot or groove into the tine of the prototype for the blade to follow, the design ensures that only the area of interest is cut and only in a controlled manner. The groove serves the additional function of allowing the blade to cut tissue without the dulling of the blade's edge.

The functionality metrics of the prototype, scalpel blade, bandage scissors and iris scissors were compared using common medical-grade textiles. The peak force, percentage of surface cut in one attempt, and number of cut attempts to completely transect an Ace™ bandage, stockinette and gauze were analyzed.

The peak force was evaluated to simulate the effort of a surgeon performing a sliding cut, where a lower force can lead to better control and decreased risk of plunging into unintended structures. Comparing peak force, the scalpel and prototype in this example performed similarly, and both required significantly less force than either the bandage or iris scissors. A scalpel is unlikely to be used for a sliding cut anywhere other than on skin due to its limited ability to control cutting depth, thereby placing deeper structures at risk of injury. Alternatively, the novel instrument prototype in this example provides a fixed and controlled cut superficial to its tine making the slide cut an ideal application for its use. The larger exerted forces observed in the bandage and iris scissors may be spuriously elevated due to the quality of the disposable instruments used in this study compared to surgical grade instruments. Disposable instruments were selected to ensure a new and consistent manufactured edge for each variable tested and to allow for reproducibility. The novel prototype of this example provides a consistently sharp cut, requiring low force at a fixed and protected depth making it ideal for settings where slide cutting techniques are deployed. Furthermore, the scalpel blade on the novel instrument can be replaced for each operation.

The percentage cut was calculated by measuring the sectioned length after one attempt and was intended to reflect the working length of the instrument. Subsequently, the total number of required attempts was recorded to quantify the overall effectiveness of the instruments under the different material properties of the Ace™ bandage, stockinette and gauze. The scalpel blade cut each material's length in one pass across 15 trials and showed its efficiency and reliability in settings where safety can be controlled externally. Similarly, the bandage scissors cut the gauze and stockinette in a single attempt and had just one instance in which the Ace™ bandage was <100% transected in one pass. This may be due to the bandage scissors being larger and more rigid than the other instruments, allowing for highly reliable cutting function but conversely less practicality in surgical settings. The iris scissors required the greatest number of attempts and cut the least amount of the intended length across all trials compared to the other instruments; this may be a result of their narrow tines and fine tips, which are best utilized for meticulous dissection through a small window. The novel surgical device of this example was superior to the iris scissors in terms of percentage cut and total number of attempts. The narrow tines of the novel device of this example allow for use in settings that require fine dissection in which bandage scissors cannot operate and with improved cutting ability compared to the iris scissors.

### Conclusion

The prototype in this example requires less force to dissect material than do iris scissors and bandage scissors, while maintaining a cutting efficacy similar to that of a traditional scalpel. Furthermore, its design qualities allow the user to dissect tissue while safely avoiding any critical blood vessels or nerve bundles. The tool in this example is a novel orthopedic instrument designed to reduce iatrogenic injuries during procedures requiring a layer-by-layer dissection approach.

While the various systems described above are separate implementations, any of the individual components, mechanisms, or devices, and related features and functionality, within the various system embodiments described in detail above can be incorporated into any of the other system embodiments herein.

The terms “about” and “substantially,” as used herein, refers to variation that can occur (including in numerical quantity or structure), for example, through typical measuring techniques and equipment, with respect to any quantifiable variable, including, but not limited to, mass, volume, time, distance, wave length, frequency, voltage, current, and electromagnetic field. Further, there is certain inadvertent error and variation in the real world that is likely through differences in the manufacture, source, or precision of the components used to make the various components or carry out the methods and the like. The terms “about” and “substantially” also encompass these variations. The term “about” and “substantially” can include any variation of 5% or 10%, or any amount—including any integer—between 0% and 10%. Further, whether or not modified by the term “about” or “substantially,” the claims include equivalents to the quantities or amounts.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range. Although the various embodiments have been described with reference to preferred implementations, persons skilled in the art will recognize that changes may be made in form and detail without departing from the spirit and scope thereof.

Although the various embodiments have been described with reference to preferred implementations, persons skilled in the art will recognize that changes may be made in form and detail without departing from the spirit and scope thereof.

What is claimed is:

1. A combination tissue manipulation and cutting device, the device comprising:
(a) a first elongate member comprising a first handle, a first jaw, and a first joint coupling structure disposed between the first handle and the first jaw;
(b) a second elongate member comprising a second handle, a second jaw, and a second joint coupling structure disposed between the second handle and the second jaw;
(c) a rotatable joint disposed at the first and second coupling structures such that the first and second elongate members are rotatable in relation to each other at the rotatable joint;
(d) a blade guide housing disposed along a length of the first elongate member, the blade guide housing comprising a distal opening, a proximal opening, and a lumen defined through the blade guide housing from the distal opening to the proximal opening; and
(e) a blade drive body slidably disposable within the lumen of the blade guide housing, the blade drive body comprising a blade attachment component at a distal end of the blade drive body.

2. The device of claim 1, wherein the blade drive body comprises a detent disposed on the blade drive body, wherein the detent extends beyond the cross-sectional width or height of the blade drive body such that the detent cannot fit within the lumen of the blade guide housing.

3. The device of claim 2, wherein the detent contacts a proximal end of the blade guide housing as the blade drive body is urged distally through the blade guide housing, thereby preventing the blade drive body from moving further distally through the blade guide housing.

4. The device of claim 1, wherein the blade drive body comprises at least one channel defined along the length of the blade drive body, and wherein the blade guide housing comprises at least one detent disposed on the blade guide housing such that the at least one detent is disposed within the at least one channel when the blade drive body is disposed within the blade guide housing.

5. The device of claim 1, wherein the blade drive body comprises a finger contact structure associated with a proximal portion of the blade drive body.

6. The device of claim 5, wherein the finger contact structure comprises ribs or protrusions.

7. The device of claim 1, wherein the blade guide housing and the blade drive body are removable.

8. The device of claim 1, further comprising a removable blade coupleable to the blade attachment component.

9. The device of claim 1, wherein the first elongate member comprises a guide slot defined within and disposed along a length of the first elongate member, wherein the blade drive body is slidably disposed adjacent to or within the guide slot.

10. The device of claim 1, wherein the blade drive body is slidable between a retracted position and a deployed position along a length of the first elongate member.

11. A combination tissue manipulation and cutting device, the device comprising:
(a) a first elongate member comprising a first handle, a first jaw, and a guide slot defined within and along a length of the first elongate member;
(b) a second elongate member comprising a second handle and a second jaw, wherein the second elongate member is rotatably coupled to the first elongate member at a rotatable joint;
(c) a blade guide housing disposed along a length of the first elongate member, the blade guide housing comprising a distal opening, a proximal opening, and a lumen defined through the blade guide housing from the distal opening to the proximal opening;
(d) a blade drive body slidably disposable within the guide slot and the lumen of the blade guide housing, the blade drive body comprising:
(i) a blade attachment component at a distal end of the blade drive body; and
(ii) a detent disposed on the blade drive body, wherein the detent extends beyond the cross-sectional width or height of the blade drive body such that the detent cannot fit within the lumen of the blade guide housing,
wherein the blade drive body is slidable between a proximal position and a distal position; and
(e) a removable blade operably coupled to the blade attachment component.

12. The device of claim 11, wherein the detent contacts a proximal end of the blade guide housing as the blade drive body is urged distally through the blade guide housing, thereby preventing the blade drive body from moving further distally through the blade guide housing.

13. The device of claim 11, wherein the blade drive body comprises a finger contact structure associated with the blade drive body, wherein the finger contact structure comprises ribs or protrusions.

14. The device of claim 11, wherein the blade guide housing and the blade drive body are removable and replaceable.

15. The device of claim 14, further comprising:
(a) at least one additional blade guide housing, wherein the at least one additional blade guide housing is interchangeable with the blade guide housing; and
(b) at least one additional blade drive body, wherein the at least one additional blade drive body is interchangeable with the blade drive body.

16. A method of manipulating and cutting tissue during a surgical procedure, the method comprising:
engaging target tissue with a surgical device, the surgical device comprising:
(a) a first elongate member comprising a first jaw and a blade guide housing attached to the first elongate member;
(b) a second elongate member rotatably coupled to the first elongate member, the second elongate member comprising a second jaw; and
(c) a blade drive body slidably disposed within the blade guide housing, the blade drive body being removably attached to a blade; and
urging the blade drive body and the blade distally through the blade guide housing into a deployed configuration such that the blade cuts the target tissue.

17. The method of claim 16, further comprising retracting the blade drive body and the blade from the deployed configuration proximally through the blade guide housing into a retracted configuration.

18. The method of claim 16, wherein the engaging the target tissue comprises grasping the tissue between the first and second jaws.

19. The method of claim 16, wherein the urging the blade drive body and the blade distally comprises placing a finger on the blade drive body and urging the blade drive body distally with the finger.

20. The method of claim 16, wherein the blade drive body comprises a detent, wherein the urging the blade drive body and the blade distally further comprises urging the blade drive body and the blade distally until the detent contacts a proximal end of the blade guide housing such that the blade drive body cannot be advanced distally any further.
